(19) United States
(12) Patent Application Publication
(10) Pub. No.: US 2024/0100120 A1
(43) Pub. Date: Mar. 28, 2024

(54) COMPOSITIONS AND METHODS FOR PREVENTING, REDUCING AND REVERSING OPIOID-INDUCED RESPIRATORY DEPRESSION

(71) Applicant: Board of Regents, The University of Taxas System, Austin, TX (US)

(72) Inventors: Glenn M. Toney, San Antonio, TX (US); Allison D. Brackley, San Antonio, TX (US)

(21) Appl. No.: 17/767,743

(22) PCT Filed: Oct. 8, 2020
(86) PCT No.: PCT/US2020/054759

§ 371 Date: Apr. 8, 2022

Related U.S. Application Data
(60) Provisional application No. 62/914,025, filed on Oct. 11, 2019.

(51) Int. Cl. A61K 38/095; A61K 31/55; A61P 11/00

(52) U.S. Cl. CPC A61K 38/095; A61K 31/55; A61P 11/00

(57) ABSTRACT

The disclosure relates to compositions and methods of preventing, reducing, reversing or treating a subject suffering from opioid toxicity, overdose or opioid-induced respiratory depression or a subject at risk for developing opioid-induced respiratory depression. The method comprises administering to a patient in need of treatment an effective amount of oxytocin or an analog thereof.

### CROSS REFERENCE TO RELATED APPLICATIONS

This application claims the benefit of priority of U.S. Provisional Application No. 62/914,025, filed Oct. 11, 2019. The content of this earlier filed application is hereby incorporated by reference herein in its entirety.

### STATEMENT REGARDING FEDERALLY SPONSORED RESEARCH

This invention was made with government support under grant no. HL08852 awarded by the National Institutes of Health. The government has certain rights in the invention.

### BACKGROUND

Opioid abuse in the United States is considered an epidemic and its causes are complex, involving many economic, social, medical, biological and other factors. The current opioid epidemic remains a serious public health challenge, despite the availability of medications that are effective in some patients (naltrexone, buprenorphine, and methadone), and attests to the desperate need for more and better approaches to treat this national emergency.

### SUMMARY

Disclosed herein are methods of preventing opioid-induced respiratory depression in a subject, the methods comprising: administering to the subject a therapeutically effective amount of oxytocin or an analog thereof and a pharmaceutically acceptable salt.

Disclosed herein are methods of reducing opioid-induced respiratory depression in a subject, the methods comprising: administering to the subject a therapeutically effective amount of oxytocin or an analog thereof and a pharmaceutically acceptable salt.

Disclosed herein are methods of reversing opioid-induced respiratory depression in a subject, the methods comprising: administering to the subject a therapeutically effective amount of oxytocin or an analog thereof and a pharmaceutically acceptable salt.

Disclosed herein are methods of preventing or mitigating one or more effects of administration of one or more opioid receptor agonists in a subject in need thereof, the methods comprising administering to the subject a therapeutically effective amount of oxytocin or an analog thereof and a pharmaceutically acceptable salt.

Disclosed herein are methods of reversing, reducing or preventing opioid-induced respiratory depression in a subject in need thereof, the methods comprising administering to the subject a therapeutically effective amount of oxytocin or an analog thereof and a pharmaceutically acceptable salt.

Disclosed herein are methods of preventing opioid receptor antagonist-induced withdrawal in a subject, the methods comprising: administering to the subject a therapeutically effective amount of oxytocin and a pharmaceutically acceptable salt.

Disclosed herein are methods of reducing opioid receptor antagonist-induced withdrawal in a subject, the methods comprising: administering to the subject a therapeutically effective amount of oxytocin and a pharmaceutically acceptable salt.

Disclosed herein are methods of treating opioid dependence and detoxification in a subject, the methods comprising: administering to the subject a therapeutically effective amount of oxytocin and a pharmaceutically acceptable salt. In some aspects, the subject is an opioid-dependent subject.

Disclosed herein are methods of preventing or reducing one or more symptoms of opioid withdrawal in a subject, the method comprising: administering to the subject a therapeutically effective amount of oxytocin or an analog thereof, an opioid receptor antagonist and a pharmaceutically acceptable salt.

Disclosed herein are pharmaceutical compositions comprising oxytocin or an analog thereof and an opioid.

Disclosed herein are pharmaceutical compositions comprising oxytocin or an analog thereof and an opioid receptor antagonist.

Other features and advantages of the present compositions and methods are illustrated in the description below, the drawings, and the claims.

### BRIEF DESCRIPTION OF DRAWINGS

FIGS. 1A-D show that systemic oxytocin can rescue and attenuate fentanyl OIRD in awake freely moving rats. For these experiments, conscious, unrestrained rats were surgically instrumented with wireless pressure transducer transmitters to monitor subpleural thoracic pressure fluctuations caused by rhythmic breathing. Inspiratory phases of breathing were monitored before and after fentanyl administration followed by oxytocin (FIG. 1A, B) (OXTN; 150 μg/kg, i.v.), which rescued OIRD. FIGS. 1C, D show simultaneous administration of fentanyl and oxytocin as a cocktail (100 μg/kg FENT+150 μg/kg OXTN, i.v.) strongly attenuated the disruption of breathing normally induced by fentanyl alone (compare to red trace in FIG. 1A. Breathing periodicity and variability are displayed as raster plots (FIG. 1A, C) and inter-event intervals (I.E.I.) (FIG. 1B, D) plotted as cubic spline histograms constructed from peak inspiratory pressure event times obtained from subpleural pressure records. For clarity, graphs have been color coordinated with baseline (sweep 1-100) shown in black, fentanyl/co-injection (sweep 101-154) shown in red, and recovery (sweep 401-500) in blue. At baseline in black, the pattern of inspiration is regular and cyclical. Fentanyl (100 μg/kg, i.v.) increased the inspiratory interval and disrupted it rhythmicity. This gradually worsened until oxytocin (150 μg/kg, i.v.) was administered at 290 s post-fentanyl to avoid lethality. Importantly, systemic oxytocin rescued lethal fentanyl OIRD in the freely moving rat and breathing recovered to exceed baseline frequency. FIGS. 1C, D show in a separate rat, the baseline pattern of inspiratory events is also regular and cyclical. Co-administration of the same lethal dose of fentanyl (100 μg/kg, i.v.) in combination with oxytocin (150 μg/kg, i.v.) largely attenuated OIRD, with the same number of breaths that spanned 290 s.

FIGS. 2A-C show that CIHHC rats have increased sensitivity to naloxone-reversible fentanyl OIRD. FIG. 2A shows the dose-response curve for normoxic (black) and CIHHC (red) plotted as inhibition of integrated phrenic nerve activity (iPNA) burst frequency averaged over a 5 min period post-fentanyl (i.v.). FIGS. 2B-C show representative iPNA traces recorded from normoxic and CIHHC rats following fentanyl (20 μg/kg, i.v.) in the absence (B) or presence (C) of naloxone (NLX; 1 mg/kg, i.v.). n=5-6/group; **p<0.01, ****p<0.0001 vs. vehicle, #p<0.05 vs. control_{dose}; ≡p<0.05 vs control IC₅₀.

FIGS. 3A-B show that PVN naloxone uncovers diminished respiratory drive in CIHHC rats. FIG. 3A shows representative iPNA trace recordings of iPNA following systemic vehicle or naloxone (NLX; 1 mg/kg, i.v.) in normoxic and CIHHC rats. FIG. 3B shows representative iPNA trace recordings of iPNA following vehicle or naloxone (NLX; 1 nmol/side, PVN) in normoxic and CIHHC rats. NLX attenuates fentanyl (20 μg/kg, i.v.) OIRD in both normoxic and CIHHC rats. n=3-6/group; *p<0.05 vs. vehicle, ##p<0.01 vs. normoxic.

FIGS. 4A-G show that systemic oxytocin attenuates fentanyl OIRD in normoxic rats and CIHHC exposed rats that serve as a sleep apnea model of enhanced OIRD sensitivity. FIG. 4A shows that systemic vehicle (VEH; saline; intravenous, i.v.) does not change integrated phrenic nerve activity (iPNA), unlike (FIG. 4B) fentanyl (FENT; 20 μg/kg, i.v.), which inhibits iPNA burst initiation in normoxic controls (black). FIG. 4C shows that systemic oxytocin (OXTN; 80 μg/kg, i.v.) pretreatment attenuates iPNA inhibition by systemic fentanyl (20 μg/kg, i.v.). In CIHHC rats (red), (FIG. 4D) systemic vehicle (VEH; saline, i.v.) induces no change of integrated phrenic nerve activity (iPNA), unlike (FIG. 4E) fentanyl (FENT; 20 μg/kg, i.v.), which inhibits iPNA burst initiation for a prolonged period of time compared to normoxic controls (compare to FIG. 4B). Systemic pretreatment by (FIG. 4F) oxytocin (OXTN; 80 μg/kg, i.v.) prevents fentanyl (20 μg/kg, i.v.)-induced iPNA burst silencing, with burst amplitude being depressed by only 30-40%. Intravenous administration of (FIG. 4G) carbetocin (CBTN; 115 μg/kg, i.v.), an analog of oxytocin, similarly prevents iPNA silencing by systemic fentanyl (20 μg/kg, i.v.). au.=arbitrary units.

FIGS. 5A-B show that oxytocin delivered directly into the hypothalamic paraventricular nucleus, a brain region that normally synthesizes oxytocin and which regulates breathing, attenuates and rescues OIRD by fentanyl in CIHHC rates. FIGS. 5A-B show representative iPNA trace recordings following bilateral PVN administration of oxytocin (OXTN; 375 pmol/side) before (FIG. 5A) or after (FIG. 5B) fentanyl (20 μg/kg, i.v.)-induced iPNA burst silencing in normoxic (black) or CIHHC (red) rats. a. u.=arbitrary units.

FIGS. 6A-B show the phenomenon of OIRD and its enhancement by sleep apnea. FIG. 6A shows the responses and summary data demonstrating that Fentanyl dose-dependently inhibits brain-driven neural inspiration, an effect exaggerated in a rat model of sleep apnea (SA). FIG. 6B shows Fentanyl delivered to a normoxic control animal (black, upper right) and an animal exposed to nocturnal CIHHC for 7 days to model human SA (red, lower right).

FIGS. 7A-B show attenuating/preventing OIRD by delivery of naloxone into the hypothalamic paraventricular nucleus (PVN). FIG. 7A shows from a representative normoxic control rat (left, black) that injection of the opioid receptor antagonist naloxone into the PVN, a brain region capable of stimulating respiration, has no effect. FIG. 7B shows that a dose of PVN-injected Fentanyl that does not reduce PNA burst amplitude in a normoxic control animal (black, left), causes a small and gradual reduction of PNA bursting in a CIHHC exposed animal (red, right), consistent with greater PVN neuronal sensitivity to fentanyl in human SA.

FIGS. 8A-B show the results of oxytocin (OXT) in an animal model of human SA. FIG. 8A shows that oxytocin in the PVN reduces OIRD, selectively in animals modeling human SA. FIG. 8B shows in a CIHHC rat modeling SA that PVN OXT rapidly and completely reverses the arrest of breathing caused by systemic fentanyl.

FIGS. 9A-E show dose-dependent fentanyl inhibition of integrated phrenic nerve activity (iPNA). FIG. 9A shows representative recordings of iPNA burst discharge from anaesthetized, ventilated rats pre-exposed to normoxia (control, black) showing responses to graded doses of fentanyl (0, 2, 20, 50 μg kg⁻¹, I.V.) Scale bar=5 min; a.u.=arbitrary units. FIGS. 9B-E show dose-response curves generated from iPNA burst data during 5 min periods at baseline (BL) and immediately after systemic administration of vehicle or fentanyl. FIG. 9B is a fentanyl dose-PNA burst frequency response curve showing that fentanyl dose-dependently reduces PNA. frequency. FIG. 9C shows the PNA burst quiescence response curve generated from onset of PNA burst arrest until the first burst to reappear post-fentanyl, where a significant period of PNA silencing occurred at the highest dose of fentanyl administered. FIG. 9D is a PNA burst amplitude dose response curve showing that the highest dose of fentanyl significantly reduced PNA burst amplitude. FIG. 9E is a PNA burst width dose response curve showing that the highest dose of fentanyl significantly reduced PNA burst width. Group data expressed as means±SEM with significance determined by two-way ANOVA with Tukey's post hoc tests. n=5-6 rats/dose/group. **p<0.01, ***p<0.001, ****p<0.0001.

FIGS. 10A-E show dose-dependent fentanyl inhibition of phrenic nerve activity (iPNA). FIG. 10A shows representative recordings of integrated phrenic nerve activity (iPNA) from anaesthetized, ventilated rats pre-exposed to normoxia (control, black) or chronic intermittent hypoxia with hypercapnia (CIHHC, red) to model human sleep apnea (SA) showing responses to graded doses of fentanyl (0, 2, 20, 50 μg kg⁻¹, I.V.). Scale bar=5 min; a.u.=arbitrary units. FIG. 10B-E shows dose-response curves generated from PNA burst data during 5 min periods at baseline (BL) and immediately after administration of vehicle or fentanyl. FIG. 10B shows fentanyl dose-PNA burst frequency response curves, where fentanyl dose-dependently reduces PNA frequency. Importantly, CIHHC produces a significant leftward shift in the dose response curve, indicating that fentanyl is more potent in a model of sleep apnea. FIG. 10C shows the quiescence response curve generated from onset of PNA burst arrest until the first burst to reappear post-fentanyl, where the period of PNA silencing that occurred at the highest dose of fentanyl administered was significantly longer in CIHHC compared to controls. FIG. 10D shows PNA burst amplitude dose response curve, which was statistically similar in controls and CIHHC. FIG. 10E shows PNA burst width dose response curve, which was statistically similar in controls and CIHHC. Group data expressed as means±SEM with significance determined by two-way ANOVA with Tukey's post hoc tests. n=5-6 rats/dose/group. *p<0.05, ***p<0.001.

FIGS. 11A-D shows that naloxone (i.v.) uncovers endogenous opioid suppression of respiratory drive in CIHHC rats. FIG. 11A shows representative phrenic nerve activity (PNA) traces with corresponding PNA burst onset-triggered PNA averages from anaesthetized, artificially ventilated rats exposed to normoxia (control, left, black) and chronic intermittent hypoxia with hypercapnia (CIHHC, right, red). Bursts were recorded at baseline and following systemic (i.v.) saline vehicle (tor. or naloxone (1 mg kg⁻¹, middle) alone or after systemic (i.v.) fentanyl (20 μg kg⁻¹) followed by naloxone (1 mg kg′, bottom). a.u.=arbitrary units. FIG. 11B shows change in PNA burst frequency within the first 5 min of vehicle (white), naloxone (grey), or naloxone post-fentanyl (black) administration in control (left) or CIHHC (right) rats compared with 5 min BL. PNA burst frequency between control and CIHHC rats was not significant different. FIG. 11C shows change in PNA burst amplitude within the first 5 min of vehicle (white), naloxone (grey), or naloxone post-fentanyl (black) administration in control (left) or CIHHC (right) rats compared with 5 min BL. PNA burst amplitude was significantly increased by naloxone alone and naloxone post-fentanyl compared to vehicle treatment in CIHHC rats and significantly increased compared to naloxone and naloxone post-fentanyl treatments in control rats. FIG. 11D shows change in PNA burst width within the first 5 min of vehicle (white), naloxone (grey), or naloxone post-fentanyl (black) administration in control (left) or CIHHC (right) rats compared with 5 min BL. PNA burst width was not significantly different between control and CIHHC rats. Significance determined by two-way ANOVA with Tukey's post hoc tests; n=5-6 rats/group. ns=not significant, **p<0.01 vs. vehicle within CIHH group, ##p<0.01 vs. same treatment in control group.

FIG. 12 shows that naloxone microinjection into the hypothalamic paraventricular nucleus (PVN, 1 nmol/side), which does not affect integrated phrenic nerve activity (iPNA) in control rats (black), increases PNA burst amplitude in CIHHC rats (green) and reveals tonic suppression of respiratory drive by endogenous opioids in the PVN. a.u.=arbitrary units, n=5/group.

FIG. 13 shows that systemic (i.v.) fentanyl (20 μg kg⁻¹) OIRD is prevented by opioid antagonism by naloxone (right, 1 nmol/side) microinjection bilaterally into the PVN of control rats (black, top) and CIHHC rats (green, bottom), but not by PVN vehicle microinjection (right). iPNA=phrenic nerve activity, a.u.=arbitrary units.

FIG. 14 shows that systemic (i.v.) fentanyl (20 μg kg⁻¹) OIRD is prevented by bilateral PVN oxytocin (1.25 μg/side) administration in CIHHC rats (green, bottom), but not in control rats (black, top). Microinjection of vehicle into the PVN is ineffective at reversing fentanyl OIRD both in control and CIHHC rats. iPNA=phrenic nerve activity, a.u.=arbitrary units.

FIG. 15 shows that systemic (i.v.) fentanyl (20 μg kg⁻¹) OIRD is prevented or rescued by bilateral PVN oxytocin (1.25 μg/side) administration in CIHHC rats (green, bottom), but not in control rats (black, top). Microinjection of vehicle is ineffective at reversing fentanyl OIRD both in control and CIHHC rats. iPNA=phrenic nerve activity, a.u.=arbitrary units.

FIG. 16 shows that systemic (i.v.) fentanyl (20 μg kg⁻¹) respiratory depression is prevented by bilateral PVN opioid antagonism by naloxone (right, 1 nmol/side) in control rats (black, top) and in rats that previously underwent bilateral excitotoxic lesioning of parvocellular PVN neurons, including oxytocin neurons, with ibotenic acid treatment (blue, bottom). Unlike PVN naloxone, PVN vehicle microinjection (right) had no effect. iPNA=phrenic nerve activity, a.u.=arbitrary units.

FIG. 17 shows that systemic (i.v.) fentanyl (20 μg kg⁻¹) respiratory depression is not prevented by oxytocin (1.25 μg/side) in the PVN of control rats (black, top) or rats that have had PVN ibotenic acid treatment (blue, bottom). Microinjection of vehicle into the PVN is ineffective at reversing fentanyl OIRD both in control and PVN ibotenic acid treated rats. iPNA=phrenic nerve activity, a.u.=arbitrary units.

FIG. 18 shows that chronic intermittent hypoxia with hypercapnia (CIHHC, red, bottom, left) increases sensitivity to respiratory depression by systemic (i.v.) treatment with buprenorphine (10 μg kg⁻¹), a synthetic opioid that is chemically distinct from fentanyl. A dose of buprenorphine that effectively suppresses iPNA bursting in CIHHC rats is ineffective in control rats (grey, top, left). Importantly, even buprenorphine at a high dose (100 μg kg⁻¹) fails to fully silence iPNA in control rats (blue, top, right). au.=arbitrary units.

FIG. 19 shows that like fentanyl, systemic (i.v.) buprenorphine (10 μg kg⁻¹) respiratory depression is prevented by naloxone (1 nmol/side) in the hypothalamic paraventricular nucleus (PVN) of control rats (grey, top) and rats that have undergone chronic intermittent hypoxia with hypercapnia (CIHHC, red, bottom). iPNA=phrenic nerve activity, a.u.=arbitrary units.

FIGS. 20A-G show that systemic oxytocin attenuates fentanyl OIRD in normoxic rats and CIHHC exposed rats that serve as a sleep apnea model of enhanced OIRD sensitivity. FIG. 20A shows that systemic vehicle (VEH; saline; intravenous, i.v.) induces no changes to integrated phrenic nerve activity (iPNA), unlike (FIG. 20B) fentanyl (FENT; 20 μg/kg, i.v.), which inhibits iPNA burst initiation in normoxic controls (black). FIG. 20C shows that systemic oxytocin (OXTN; 80 μg/kg, i.v.) pretreatment attenuates iPNA inhibition by systemic fentanyl (20 μg/kg, i.v.). FIG. 20D shows that in CIHHC rats (red), systemic vehicle (VEH; saline, i.v.) induces no change of integrated phrenic nerve activity (iPNA), unlike fentanyl (FIG. 20E) (FENT; 20 μg/kg, i.v.), which inhibits iPNA burst initiation for a prolonged period of time com-pared to normoxic controls (compare to FIG. 20B). FIG. 20F shows that systemic pretreatment by oxytocin (OXTN; 80 μg/kg, i.v.) prevents fentanyl (20 μg/kg, i.v.)-induced iPNA burst silencing, with burst amplitude being depressed by 30-40%. FIG. 20G shows that intravenous administration of carbetocin (CBTN; 115 μg/kg, i.v.), an analog of oxytocin, similarly pre-vents iPNA silencing by systemic fentanyl (20 μg/kg, i.v.). a.u.=arbitrary units.

FIGS. 21A-D shows that oxytocin can act in the hypothalamic paraventricular nucleus (PVN) to attenuate and rescue fentanyl-induced OIRD. In CIHHC rats, bilateral PVN nanoinjection of vehicle (VEH; PBS) neither (FIG. 21A) attenuates nor (FIG. 21B) rescues inhibition of integrated phrenic nerve activity (iPNA) by systemic fentanyl (FENT; 20 μg/kg; intravenous, i.v.). On the other hand, bilateral nanoinjection of oxytocin (OXTN; 375 pmol/side) can both (FIG. 21C) attenuate and (FIG. 21D) rescue iPNA inhibition by systemic fentanyl (20 μg/kg, i.v.). a.u.=arbitrary units.

FIGS. 22A-D show that systemic oxytocin can rescue and attenuate fentanyl OIRD in awake freely moving rats. Fentanyl administration was followed by (FIGS. 22A, B) oxytocin (OXTN; 150 μg/kg, i.v.), which rescued OIRD. Simultaneous administration of (FIGS. 22C,D) fentanyl and oxytocin as a cocktail (100 μg/kg FENT+150 μg/kg OXTN, i.v.) strongly attenuated the disruption of breathing normally induced by fentanyl alone (compare to red trace in FIG. 22A). Breathing periodicity and variability are displayed as (FIGS. 22A,C) raster plots and (FIGS. 22B,D) inter-event interval (I.E.I.) cubic spline histograms constructed from peak inspiratory pressure event times obtained from subpleural pressure records. For clarity, graphs have been color coordinated with baseline (sweep 1-100) shown in black, fentanyl/coinjection (sweep 101-154) shown in red, and recovery (sweep 401-500) in blue. At baseline in black, the pattern of inspiration is regular and cyclical. FIGS. 22C, D show that in a separate rat, the baseline pattern of inspiratory events is also regular and cyclical. Co-administration of the same lethal dose of fentanyl (100 μg/kg, i.v.) in combination with oxytocin (150 μg/kg, i.v.) largely attenuated OIRD, with the same number of breaths that spanned 290 s.

FIGS. 23A-D show that reversal of fentanyl OIRD by systemic oxytocin does not depend on cross-activation of vasopressin receptors in rats. FIG. 23A shows that systemic vehicle (VEH; saline; intravenous, i.v.; TOP LEFT) does not reverse fentanyl (FENT; 20 μg/kg, i.v.) silencing of integrated phrenic nerve activity (iPNA) unlike oxytocin (OXTN; 5 μg/kg, i.v.; BOTTOM LEFT), which robustly reinitiates iPNA burst firing in rats. A nearly equimolar dose of vasopressin (VP; 5 μg/kg, i.v.; TOP RIGHT) does not reverse fentanyl. Non-peptide OXTNR agonist/VP1aR antagonist WAY-267,474 (50 μg/kg, i.v.; BOTTOM RIGHT) reverses systemic fentanyl OIRD. FIG. 23B shows that the dose-response curve for OXTN reveals bell-shaped response curve, indicating reduced efficacy of OXTN at a very high dose (50 μg/kg, i.v.) compared to lower doses. High dose OXTN efficacy is restored by the vasopressin V1aR antagonist (Phenylacl-D-Tyr(Et)2,Lys6,Arg8,des-Gly9)-vasopressin trifluoroacetate (1.1 μg/kg, i.v.; V1aRX), indicating that cross-activation of V1aR explains reduced efficacy of high dose OXTN to reverse OIRD. Furthermore, antagonism by V1aRX significantly enhances reversal of OXTN at 5 μg/kg. Moreover, OXTN receptor antagonism with atosiban (20 μg/kg, i.v.; OXTRX) prevents OXTN (5 μg/kg) reversal of FENT OIRD. † p<0.01, ‡p<0.001, § p<0.0001; black symbols vs. VEH; red symbols vs. control at specific dose; blue symbol vs. control at specific dose. FIG. 23C shows that like OXTN, systemic administration of the non-peptide OXTNR agonist WAY-267,474 (50 μg/kg, i.v.) reverses systemic fentanyl (20 μg/kg, i.v.) OIRD, which is not reversed by systemic vehicle (2.5% DMSO in saline; i.v.). § p<0.0001 vs. Vehicle. n=4-6/group. a.u.=arbitrary units.

FIGS. 24A-B show reversal of fentanyl OIRD by systemic oxytocin in mice. FIG. 24A shows that systemic vehicle (VEH; saline; intravenous, i.v.; LEFT) does not reverse fentanyl (FENT; 10 μg/kg, i.v.) inhibition of spontaneously breathing in anesthetized mice unlike (FIG. 24B) oxytocin (OXTN; 100 μg/kg, i.v.; RIGHT), which reverses systemic fentanyl OIRD. BPM=breaths per minute.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease, disorder or condition. The term “patient” includes human and veterinary subjects. In some aspects of the disclosed methods, the “patient” has been diagnosed with a need for treatment for preventing, reversing or reducing opioid induced respiratory depression, such as, for example, prior to the administering step. In some aspects, the patient has obstructive sleep apnea or sleep apnea.

As used herein, the term “side effect” refers to adverse effects or toxic effects produced by a drug including but not limited to on a tissue or organ system. Such conditions, for example, can include sedation, dizziness, nausea, vomiting, constipation, hyperalgesia, immunologic and hormonal dysfunction, muscle rigidity, myoclonus, bowel dysfunction (e.g., anorexia, gastro-oesophageal reflux, delayed digestion, abdominal pain, bloating) respiratory depression and death.

As used herein, the terms “opioid toxicity” or “opioid overdose” refers to the effects of opioid drugs that are toxic to a subject, resulting in effects that may be mild, moderate or severe including but not limited to respiratory depression or ventilatory depression, hypoxia, loss of consciousness, decreased respiratory rate, decreased respiratory depth, apnea, delirium, hypotension, bradycardia, decreased body temperature, urinary retention, pupil miosis, sedation, dizziness, nausea, vomiting, constipation, hyperalgesia, immunologic and hormonal dysfunction, muscle rigidity, myoclonus, and bowel dysfunction. Opioid toxicity can be assessed by any appropriate method known to a clinical physician or skilled artisan. For example, opioid toxicity can be assessed by performing a central nervous system review by assessing for confusion, altered mental state, excessive drowsiness, lethargy, stupor, slurred speech (new onset), hypoventilation, shortness of breath, apnea, hypoxia, and/or hypercapnia; and/or cardiac review by assessing for bradycardia, hypotension, and/or shock.

The term “overdose” refers to a subject that takes a dose larger than originally intended that results in one or more adverse effects.

As used herein, the term “treating” refers to partially or completely alleviating, ameliorating, relieving, delaying onset of, inhibiting or slowing progression of, reducing severity of, and/or reducing incidence of one or more signs or symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be toxicity, abuse or an overdose relating to the administration of one or more opioids.

As used herein, the phrase “non-dependent” refers to a subject that has or has not taken an opioid at least once and has or has not have become opioid dependent but is not opioid dependent prior to the instance for which treatment is sought.

As used, herein, the phrase “opioid experienced” refers to a subject that has taken an opioid at least once prior to the instance for which treatment is sought. In some aspects, a subject can be opioid experienced and non-dependent suffering from opioid toxicity or opioid overdose. In some aspects, a subject can be opioid experienced and opioid dependent suffering from opioid toxicity or opioid overdose. In some aspects, opioid experienced and non-dependent subjects can be those subjects who have used opioid drugs in the past and have not developed tolerance or dependence to the opioid drugs and can be treated for opioid toxicity or overdose. In some aspects, opioid experienced and opioid-dependent subjects can be those subjects who have used opioid drugs in the past and might have developed tolerance or dependence to the opioid drugs and can be treated for opioid toxicity or overdose.

As used herein, the term “prevent” or “preventing” refers to preventing in whole or in part, or ameliorating or controlling.

As used herein, the terms “dependent”, “drug dependence” and “opioid dependent” refer to an adaptive state that develops from repeated (two or more) drug administration, and which results in withdrawal upon cessation of drug use. “Physical dependence” refers to an altered physiological state produced by the repeated administration of the tolerance-forming drug that necessitates the continued administration of the drug, requiring more of it to achieve its effects, eliciting drug specific physical or mental symptoms if drug use is discontinued manifesting as withdrawal or abstinence syndrome.

As used herein, the term “opioid substance misuse and abuse” refers to the misuse, in the case of prescription opioid medications, and abuse, in the case of illicit opioid substances, of opioid substances that lead to problematic medical or social patterns within a given culture. This can manifest as the compulsive seeking and usage of opioid substances, whether prescription and/or illicit.

The current state of the art is to reverse opioid-induced respiratory depression by systemic (intravenous) administration of the mu opioid-receptor antagonist, naloxone. A major disadvantage of this approach is that naloxone interferes with opioid-induced analgesia (attenuation of pain perception). Therefore, a major advantage of the compositions and methods disclosed herein is that the administration of oxytocin or an analog thereof does not interfere with opioid-induced analgesia. Thus, the pain killing effects of opioid analgesic drugs can be maintained while selectively reversing or preventing opioid-induced respiratory depression and death using the compositions and methods disclosed herein. In fact, oxytocin has been reported to have pain killing (analgesic) properties and could actually allow pain management to be achieved with a smaller dose of opioid analgesic.

Described herein are compounds and compositions (e.g. oxytocin and analogs thereof) that acts rapidly to reduce or prevent as well as reverse depression of respiration caused by systemic opioid pain killers. Opioid induced respiratory depression (OIRD) is the leading cause of death due to opioid drugs and is the major cause of the opioid death epidemic underway in the United States. A major advantage of oxytocin is that it is produced normally in humans and does not interfere with the pain killing properties of opioids. Interference with pain killing properties of opioids is the major drawback to the currently available treatment for OIRD—naloxone (Narcan®). In fact, oxytocin enhances the pain killing effects of opioids, thereby providing a further benefit in allowing use of lower doses of opioids and further mitigating the prevalence of lethal OIRD.

The compositions and methods disclosed herein can be used: (1) to prevent or reverse post-operative opioid induced respiratory depression with maintenance of pain relief (analgesia); and (2) to prevent or reverse respiratory-depression related death due to opioid overdose. And, because oxytocin has analgesic actions, its use could mitigate or prevent opioid death due to respiratory depression directly as well as indirectly aid the same endpoint by allowing a smaller dose of opioid to be used for pain management.

Opioids bind to opioid receptors to produce analgesia. Opioids are prescribed following surgery, injury, or for certain health conditions. Although opioids are effective at treating pain, prescription opioids are associated with risks and side effects, even when taken as directed. Said risks and side effects include but are not limited to: tolerance, physical dependence, addiction/substance use disorder, increased sensitivity to pain, constipation, itching and sweating, nausea, vomiting, and dry mouth, sleepiness and dizziness, confusion, low levels of testosterone, and respiratory depression.

Animal studies have demonstrated that under stress, opioids inhibit oxytocin release from hypothalamic neurons. As described herein, in the presence of increased CSF opioids, systemic naloxone induces systemic oxytocin release from the posterior pituitary with concurrent rise in CSF oxytocin.

Human studies have revealed that sleeping disorders exacerbate opioid-induced respiratory events. For instance, one-third of post-operative patients with opioid-induced postoperative respiratory depression have sleep apnea, and one-half of post-operative patients who died of opioid-induced respiratory depression had sleep apnea. Thus, sleep apnea patients are at an increased risk of requiring naloxone to reverse adverse respiratory events. Additionally, studies in human show that sleep apnea symptoms improve with naloxone treatment. Currently, no models of sleep apnea-related OIRD exist.

Chronic intermittent hypoxia with hypercapnia (CIHHC) is a rat model of sleep apnea. CIHHC rats have reduced optogenetically stimulated oxytocin release from PVN terminals in the brainstem. In human sleep apnea patients, oxytocin treatment improved frequency of hypopnea as well as apnea and hypopnea duration and related arousal. As disclosed herein CIHHC increases sensitivity to fentanyl respiratory depression.

Compositions

As disclosed herein, oxytocin can be useful for preventing, reducing, or reversing opioid induced respiratory depression. As disclosed herein, oxytocin analogs can be useful for preventing, reducing, or reversing opioid inducing respiratory depression. As disclosed herein, oxytocin and oxytocin analogs can be useful for preventing or reducing opioid withdrawal and dependence or for detoxification when combined with an opioid receptor antagonist. Also as disclosed herein, oxytocin and oxytocin analogs can be useful for preventing, reducing or reversing opioid induced respiratory depression when combined with an opioid receptor antagonist. Examples of oxytocin analogs include, but are not limited to peptides such as carbetocin, demoxytocin, lipo-ocytocin-1, and merotocin, and non-peptides such as WAY-267,474 and TC OT 39. In some aspects, the oxytocin analog can be carbetocin, demoxytocin, lipo-ocytocin-1, merotocin, WAY-267,474, or TC OT 39.

Oxytocin and oxytocin analogs can be useful in the treatment, reduction, reversal and/or prevention of opioid-induced respiratory depression, opioid overdose or opioid toxicity, the treatment, prevention or reversal of respiratory depression, ameliorating one or more signs or symptoms of opioid toxicity or opioid toxicity, and the prevention or mitigation of one or more effects of one or more opioid receptor agonists, resulting from opioids including but not limited to alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl and derivatives, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, homologues thereof, analogs thereof and any mixtures thereof.

Oxytocin and oxytocin analogs can also be useful in the treatment, reduction, and/or prevention of opioid receptor antagonist-induced withdrawal symptoms in a subject, ameliorating one or more signs or symptoms of opioid antagonism in a subject, and the prevention or mitigation of one or more effects of opioid antagonism in a subject. In some aspects, the opioid-induced withdrawal symptoms, the one or more signs or symptoms of opioid antagonism or the effects of opioid antagonism can be the result of the administration of one or more opioid receptors antagonists including, but not limited to, naloxone, naloxone-buprenorphine (Suboxone®), naltrexone, homologues thereof, analogs thereof and any mixtures thereof. Oxytocin and oxytocin analogs can also be useful in the treatment and/or reduction of opioid administration in an opioid-dependent subject, ameliorating one or more signs or symptoms of substance use treatment (e.g., the administration naltrexone can be administered to a subject to treat other types (non-opioid) of chemical dependence), and the prevention or mitigation of one or more effects of opioid antagonism. In some aspects, treating or reducing opioid administration in an opioid-dependent subject, the one or more signs or symptoms of substance use treatment and the one or more effects of opioid antagonism can be the result of the administration of one or more opioid receptor antagonists including but not limited to naloxone, naloxone-buprenorphine (Suboxone®), naltrexone, homologues thereof, analogs thereof and any mixtures thereof.

Examples of signs or symptoms of opioid withdrawal include but are not limited to anxiety, nausea, vomiting, abdominal pain, restlessness, sweating, pain (e.g., in muscles), dilated pupils or water eyes, cramping abdominal pain, fast heart rate, excessive yawning, goose bumps, insomnia and tremor.

In some aspects, the opioid receptor agonist can be a full agonist (e.g., heroin, oxycodone, methadone, hydrocodone, morphine, opium, fentanyl, etc.). A full agonist is a drug that activates a given receptor fully resulting in the full opioid effect. In some aspects, the opioid receptor can be a partial agonist (e.g., buprenorphine, tramadol). A partial agonist is a drug that binds to and activates a given receptor but has only partial efficacy relative to a full agonist.

Oxytocin and oxytocin analogs can also be useful for the treatment of non-dependent subjects suffering from or at risk for developing opioid toxicity or overdose or opioid-induced respiratory depression. In some aspects, those subjects who have used any opioid in the past and have not developed tolerance or dependence to any of the opioids can be treated for opioid toxicity or overdose or opioid-induced respiratory depression. Oxytocin and oxytocin analogs can also be useful for the treatment of subjects that have not used opioids in the past and are first-time users suffering from opioid toxicity or overdose or opioid-induced respiratory depression. Oxytocin and oxytocin analogs can also be useful for the treatment of subjects that are experienced opioid users suffering from opioid toxicity or overdose or opioid-induced respiratory depression. Oxytocin and oxytocin analogs can be useful for the treatment of opioid abuse or protecting or preventing opioid-induced respiratory depression or opioid overdose in subjects that can be an experienced opioid user that is not actively using opioids. Oxytocin and oxytocin analogs can also be useful for the treatment of subjects that are non-dependent but experienced opioid users suffering from opioid toxicity or overdose or opioid-induced respiratory depression. Oxytocin and oxytocin analogs can also be useful for the treatment, reduction, and/or prevention of opioid receptor antagonist-induced withdrawal symptoms in dependent and non-dependent subjects. In some aspects, the dependent and non-dependent subjects received one or more opioid receptor antagonists prior to the administration of oxytocin or an oxytocin analog. In some aspects, the subject was administered one or more opioid receptor antagonist for the treatment or prevent of opioid toxicity or overdose or opioid-induced respiratory depression. Oxytocin and oxytocin analogs can also be useful for the treatment of withdrawal symptoms associated with the administration of one or more opioid receptor agonists and antagonists in a subject that has not used an opioid in the past and is a first time user suffering from opioid toxicity or overdose or opioid-induced respiratory depression. Oxytocin and oxytocin analogs can also be useful for the treatment of withdrawal symptoms associated with the administration of one or more opioid receptor antagonists in a subject that has not used an opioid in the past and is a first time user suffering from opioid toxicity or overdose or opioid-induced respiratory depression. Oxytocin and oxytocin analogs can also be useful for the treatment of withdrawal in subjects that are actively using opioids with a history of opioid substance abuse. Oxytocin and oxytocin analogs can also be useful for managing opioid use and detoxification by opioid receptor agonists and antagonists in subjects that are dependent on opioids or those that with a history of opioid substance abuse. Oxytocin and oxytocin analogs can also be useful for managing opioid use and detoxification by opioid receptor antagonists in subjects that are dependent on opioids or those that with a history of opioid substance abuse.

In some aspects, the time period of therapeutic effectiveness of oxytocin and oxytocin analogs from a single (or multiple) dose(s) administration can be within minutes. The time period of therapeutic effectiveness of oxytocin and oxytocin analogs from a single (or multiple) dose(s) administration can last from about 1 minute to 60 minutes, 1 hour to 24 hours or 1 day to 7 days to over 1 week. In some aspects, a time period of therapeutic effectiveness of oxytocin can be between 1 to 5, 5 to 10, 10 to 15, 15 to 20, 20 to 25, 25 to 30, 30 to 35, 35 to 40, 40 to 45, 45 to 50, 50 to 55, 55 to 60 minutes, 1 to 4, 4 to 8, 8 to 12, 12 to 16, 16 to 20, 20 to 24 hours, 1 to 2, 2 to 3, 3 to 4, 4 to 5, 5 to 6, 6 to 7 days, 1 to 2 weeks or longer or any time period in between.

In some aspects, oxytocin and oxytocin analogs can be used in combination with other therapeutic drugs used to treat subjects suffering from or at risk for developing opioid toxicity, overdose or opioid-induced respiratory depression. In some aspects, oxytocin and oxytocin analogs can be used in combination with other therapeutic drugs used to prevent or reduce one or more symptoms of opioid withdrawal in a subject. In some aspects, the opioid withdrawal can be withdrawal of one or more opioid receptor agonists. In some aspects, the opioid withdrawal can be induced by the administration of one or more opioid receptor antagonists. For example, in some aspects, oxytocin and oxytocin analogs can be administered with other opioid receptor antagonists. In some aspects, disclosed herein are compositions comprising oxytocin or an analog thereof and an opioid receptor antagonist. Examples of opioid receptor antagonists include but are not limited to naloxone, naltrexone, methylnaltrexone, nalmefene, nalodeine, samidorphan and analogs thereof.

In some aspects, oxytocin and oxytocin analogs can be used in combination with other therapeutic drugs to treat, for example, pain or opioid management and detoxification (e.g., opioid withdrawal signs or symptoms), to prevent, reduce, reverse or treat opioid toxicity, overdose or opioid-induced respiratory depression. In some aspects, the oxytocin and oxytocin analogs can be administered before, after or simultaneously with the therapeutic agent. In some aspects, the therapeutic drug can be an opioid. As used herein, the term “opioid management” refers to the use of prescribed opioid medications under the medical guidance of physicians and healthcare professionals. As used herein, the term “detoxification” refers to the process of ridding the body of a drug or its metabolites, which can be done through medically-assisted management of withdrawal symptoms.

### Methods of Treatment

Disclosed herein, are methods of treating opioid toxicity in a subject comprising administering to the subject a therapeutically effective amount of oxytocin or an oxytocin analog.

Disclosed herein, are methods of preventing opioid toxicity in a subject comprising administering to the subject a therapeutically effective amount of oxytocin or an oxytocin analog.

Disclosed herein, are methods of treating opioid overdose in a subject comprising administering to the subject a therapeutically effective amount of oxytocin or an oxytocin analog.

Disclosed herein, are methods of preventing opioid overdose in a subject comprising administering to the subject a therapeutically effective amount of oxytocin or an oxytocin analog.

Disclosed herein, are methods of treating opioid-induced respiratory depression in a subject comprising administering to the subject a therapeutically effective amount of oxytocin or an oxytocin analog.

Disclosed herein, are methods of preventing opioid-induced respiratory depression in a subject comprising administering to the subject a therapeutically effective amount of oxytocin or an oxytocin analog.

Disclosed herein, are methods of reducing opioid-induced respiratory depression in a subject comprising administering to the subject a therapeutically effective amount of oxytocin or an oxytocin analog.

Disclosed herein, are methods of reversing opioid-induced respiratory depression in a subject comprising administering to the subject a therapeutically effective amount of oxytocin or an oxytocin analog.

Disclosed herein, are methods of ameliorating one or more signs or symptoms of opioid toxicity, opioid overdose or opioid-induced respiratory depression in a subject comprising administering to the subject a therapeutically effective amount of oxytocin or an oxytocin analog.

Disclosed herein, are methods of preventing or mitigating one or more effects of administration of one or more opioid receptor agonists in a subject in need thereof comprising administering to the subject a therapeutically effective amount of oxytocin or an oxytocin analog.

Disclosed herein, are methods of reversing or preventing opioid-induced respiratory depression in a subject in need thereof comprising administering to the subject a therapeutically effective amount of oxytocin or an oxytocin analog.

Disclosed herein, are methods of preventing opioid withdrawal in a subject comprising administering to the subject a therapeutically effective amount of oxytocin or an oxytocin analog.

Disclosed herein, are methods of preventing opioid receptor antagonist-induced withdrawal in a subject comprising administering to the subject a therapeutically effective amount of oxytocin or an oxytocin analog.

Disclosed herein, are methods of reducing opioid receptor antagonist-induced withdrawal in a subject comprising administering to the subject a therapeutically effective amount of oxytocin or an oxytocin analog.

Disclosed herein, are methods of treating opioid dependence and detoxification in an opioid-dependent subject comprising administering to the subject a therapeutically effective amount of oxytocin or an oxytocin analog.

Disclosed herein, are methods of preventing or reducing one or more symptoms of opioid withdrawal in a subject. In some aspects, the methods can comprise administering to the subject a therapeutically effective amount of oxytocin or an analog thereof, an opioid receptor antagonist and a pharmaceutically acceptable salt.

In any of the methods disclosed herein, the methods can comprise identifying a subject in need of treatment. The methods can also include administering to the subject a therapeutically effective amount of oxytocin or an oxytocin analog and a pharmaceutically acceptable salt. In some aspects, the methods can also include administering to the subject a therapeutically effective amount of oxytocin or an oxytocin analog, an opioid receptor antagonist, and a pharmaceutically acceptable salt.

In some aspects, oxytocin or an oxytocin analog can be administered to a subject via constant delivery of low doses, for example, by a sustained release formulation in a method of preventing opioid toxicity, preventing opioid overdose, preventing opioid-induced respiratory depression or preventing one or more effects of administration of one or more opioid receptor agonists.

In some aspects, oxytocin or an oxytocin analog can be administered to a subject via a rapid delivery, for example, by injection or nasal administration, in a method of treating opioid toxicity, treating opioid overdose, treating opioid-induced respiratory depression, treating or ameliorating one or more signs or symptoms of opioid toxicity or opioid overdose or reversing opioid-induced respiratory depression.

In some aspects, a therapeutically effective amount of a pharmaceutical composition comprising oxytocin or an oxytocin analog and a pharmaceutically acceptable carrier can be administered to the subject. In some aspects, the opioid toxicity or overdose or opioid-induced respiratory depression or one or more effects of administration of one or more opioid receptor agonists results from opioid administration to a non-dependent subject. In some aspects, the subject can be an experienced opioid user. In some aspects, the subject can be a non-dependent, experienced opioid user. In some aspects, the administration of oxytocin or an oxytocin analog can reduce one or more signs or symptoms of opioid toxicity or overdose. In some aspects, oxytocin or an oxytocin analog can ameliorate one or more signs or symptoms of opioid toxicity or overdose. In some aspects, oxytocin or an oxytocin analog can prevent or mitigate one or more effects of administration of one or more opioid receptor agonists. In some aspects, oxytocin or an oxytocin analog can prevent, reduce or reverse opioid-induced respiratory depression. In some aspects, oxytocin or an oxytocin analog can reverse and/or prevent one or more signs or symptoms of opioid toxicity or overdose, and in some cases, all of the symptoms of opioid toxicity or overdose, that are associated with actions at μ-opioid receptors. In some aspects, a therapeutically effective amount of oxytocin or an oxytocin analog can produce a long lasting relief from opioid toxicity, opioid overdose, one or more signs or symptoms of opioid toxicity or overdose, opioid-induced respiratory depression, or one or more effects of administration of one or more opioid receptor agonists. In some aspects, the long lasting relief can last at least 7 days or longer. In some aspects, one or more of the symptoms of opioid toxicity or overdose can be reduced over a period of at least 1 to about 60 minutes. In some aspects, one or more of the symptoms of drug toxicity or overdose can be reduced over a period of at least 1 hour, at least 2 hours, at least 3 hours, at least 4 hours, at least 8 hours, at least 12 hours, at least 16 hours, at least 20 hours, at least 24 hours or more. In some aspects, one or more of the symptoms of opioid toxicity or overdose can be reduced over a period of at least 1 to about 24 hours. In some aspects, one or more of the symptoms of drug toxicity or overdose can be reduced over a period of at least 1 day, at least 2 days, at least 3 days, at least 4 days, at least 5 days, at least 6 days, at least 7 days or more. In some aspects, one or more of the symptoms of opioid toxicity or overdose can be reduced over a period of at least 1 to about 7 days. In some aspects, one or more of the symptoms of drug toxicity or overdose can be reduced over a period of at least 1 week, at least 2 weeks or more. In some aspects, one or more of the symptoms of opioid toxicity or overdose can be reduced over a period of at least 1 to about 2 weeks. In some aspects, the one or more signs or symptoms of opioid toxicity or overdose can be sedation, dizziness, nausea, vomiting, confusion, loss of consciousness, constipation, muscle rigidity, miosis, myoclonus, and respiratory depression.

The pharmaceutical compositions described herein can be formulated to include a therapeutically effective amount of oxytocin or an oxytocin analog. In some aspects, oxytocin or an oxytocin analog can be contained within a pharmaceutical formulation. In some aspects, the pharmaceutical formulation can be a unit dosage formulation. In some aspects, oxytocin or an oxytocin analog can be administered on an as-needed basis.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to one or more side effects associated with opioid use.

The pharmaceutical compositions described herein can be administered to the subject (e.g., a human patient) in an amount sufficient to delay, reduce, prevent or reverse the onset or duration of opioid toxicity or overdose or respiratory depression in a subject. Accordingly, in some aspects, the patient can be a human patient. In some aspects, the patient can have sleep apnea or obstructive sleep apnea. In therapeutic applications, compositions can be administered to a subject (e.g., a human patient) already expressing or diagnosed with one or more opioid toxic symptoms in an amount sufficient to at least partially improve a sign or symptom or to inhibit the progression of (and preferably arrest) the symptoms of the condition, its complications, and consequences. An amount adequate to accomplish this is defined as a therapeutically effective amount. A therapeutically effective amount of a pharmaceutical composition can be an amount that achieves a cure or reverses or reduces one or more signs or symptoms of opioid toxicity or opioid overdose, but that outcome is only one among several that can be achieved. As noted, a therapeutically effective amount includes amounts that provide a treatment in which the onset, progression or expression of one or more of the side effects associated with opioid use or toxicity or overdose or opioid-induced respiratory depression or opioid receptor antagonist-induced withdrawal is delayed, hindered, or prevented, or the one or more signs or symptoms associated with opioid use or toxicity or overdose or opioid-induced respiratory depression or preventing opioid receptor antagonist-induced withdrawal is reduced, ameliorated or reversed. One or more of the symptoms can be less severe. Recovery can be accelerated in an individual who has been treated. A therapeutically effective amount can also include amounts that will not compromise an opioid-mediated analgesia.

Amounts effective for this use can depend on the severity of the symptoms of opioid toxicity or overdose or respiratory depression and the weight and general state and health of the subject, but generally range from about 0.1-350 mg of an equivalent amount of the oxytocin or an oxytocin analog per dose per subject.

The total effective amount of a oxytocin or an oxytocin analog as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions and sustained release formulations sufficient to maintain therapeutically effective concentrations in the blood are also within the scope of the present disclosure.

The therapeutically effective amount or dosage of the oxytocin or an oxytocin analog used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of side effects of the opioid agonists), the age and physical characteristics of the subject and other considerations known to those of ordinary skill in the art. Dosages of oxytocin can be in the range of 0.001 mg to 5 mg per kilogram of the subject's body weight. In some aspects, the dosage of oxytocin can be 1, 10, 100, 200, 300, or 350 mg total. In some aspects, the dosage of oxytocin can be 0.001 to 5 mg/kg. In some aspects, oxytocin or an oxytocin analog can be administered orally, intravenously, intramuscularly, subcutaneously, or intranasally. The side effects (e.g., respiratory depression) of opioid receptor agonists can be reversed by 0.32 mg/kg oxytocin subcutaneously in rhesus monkeys that received heroin (0.1-1.0 mg/kg). In some aspects, a single administration of oxytocin or an oxytocin analog can reverse opioid-induced respiratory depression in a subject. In some aspects, a single administration of oxytocin or an oxytocin analog can reverse opioid overdose in a subject. In some aspects, a single administration of oxytocin or an oxytocin analog can prevent opioid overdose in a subject for one or more days. In some aspects, a single administration of oxytocin or an oxytocin analog can prevent respiratory depression in a subject for one or more days. In some aspects, a single administration of oxytocin or an oxytocin analog can prevent or reduce opioid receptor antagonist withdrawal in a subject for one or more days. In some aspects, multiple administrations of oxytocin or an oxytocin analog can reverse opioid-induced respiratory depression in a subject. In some aspects, multiple administrations of oxytocin or an oxytocin analog can reverse opioid overdose in a subject. In some aspects, multiple administrations of oxytocin or an oxytocin analog can prevent opioid overdose in a subject for one or more days. In some aspects, multiple administrations of oxytocin can prevent respiratory depression in a subject for one or more days. In some aspects, multiple administrations of oxytocin or an oxytocin analog can prevent or reduce opioid receptor antagonist withdrawal in a subject for one or more days. The equivalent range of oxytocin doses in humans assuming a body weight of 70 kg can be about 0.001-5 mg/kg total. In some aspects, the therapeutically effective amount of oxytocin or an oxytocin analog can be between 0.001-5 mg/kg of body weight or any amount in between. In some aspects, smaller therapeutically effective doses may retain the ability to reverse one or more side effects (e.g., behaviorally disruptive effects) of opioid receptor agonists.

In some aspects, oxytocin and an analog thereof can be administered using any of the following strengths: 10 units/mL; 30 units/500 mL-0.9%; 30 units/500 mL-D5% LR; 30 units/500 mL-NaCl 0.45%; 20 units/1000 mL-0.9%; 40 units/1000 mL-0.9%; 10 units/1000 mL-D5% LR; 20 units/1000 mL-D5% LR; 10 units/1000 mL-LR; 20 units/1000 mL-LR; 30 units/500 mL-LR; 10 units/1000 mL-0.9%; 10 units/500 mL-LR; 20 units/1000 mL-D5%; 30 units/500 mL-5%; 10 units/500 mL-5%; 20 units/500 mL-5%; 40 units/500 mL-5%; 10 units/1000 mL-D5%; 30 units/1000 mL-D5%; 40 units/1000 mL-D5%; 10 units/500 mL-0.9%; 20 units/500 mL-0.9%; 40 units/500 mL-0.9%; 30 units/1000 mL-0.9%; 10 units/500 mL-D5% LR; 20 units/500 mL-D5% LR; 40 units/500 mL-D5% LR; 20 units/500 mL-LR; 30 units/1000 mL-LR; 15 units/250 mL-LR; 60 units/1000 mL-NaCl 0.9%; 15 units/250 mL-D5%; 10 units/1000 mL-D5% with 0.45% NaCl; 20 units/1000 mL-D5% with 0.45% NaCl; units/1000 mL-D5% with 0.45% NaCl; 15 units/250 mL-D5% with 0.225% NaCl; 30 units/1000 mL-D5% LR; 20 units/1000 mL-D5% with 0.9% NaCl; 30 units/500 mL-D5% with 0.9% NaCl; 5 units/500 mL-LR; 40 units/1000 mL-LR; 15 units/250 mL-NaCl 0.9%; 30 units/500 mL-NaCl 0.9%.

In some aspects, an opioid receptor antagonist can be administered between 0.01 mg to 500 mg. In some aspects, Narcan® (naloxone hydrochloride injection USP) can be administered via intravenous, intramuscular, or subcutaneous administration, and is available as multiple dose vials in 0.4 mg/mL or 1 mg/mL in 10 mL multiple dose vial boxes of one. Narcan® (naloxone hydrochloride injection USP) is also available in preservative-free ampules of 0.02 mg/mL and 1 mg/mL in 2 mL unit dose ampule boxes of 10, as well as 0.4 mg/mL in 1 mL unit dose ampule boxes of 10. The dosages can range from 0.02 mg/mL to 2 mg/mL depending on the source. In some aspects, any of the dosages can be diluted for infusion in 0.9% NaCl or d5% w solutions. For opioid dependence, in some aspects, naltrexone can be administered in an initial 25 mg dose tablet, followed by 50 mg daily thereafter. In some aspects, naltrexone can be administered using an intramuscular dose of 380 mg every 4 weeks for maintenance of abstinence. In some aspects, 300 mg per day of naltrexone can be administered to subjects. Any of the oxytocin or oxytocin analogs (including formulations and dosages) described herein can be administered in combination with any of the opioid receptor antagonists described herein at dosage strengths ranging from 0.18 and 4 mg/mL naloxone and up to 380 mg for naltrexone. In some aspects, the dosage and/or concentration range of naloxone or naltrexone can vary depending the route of administration and the formulation.

Pharmaceutical Compositions

As disclosed herein, are pharmaceutical compositions, comprising oxytocin or analogs thereof and a pharmaceutical acceptable carrier described herein. Also disclosed herein, are pharmaceutical compositions, comprising oxytocin or analogs thereof, an opioid receptor antagonist, and a pharmaceutical acceptable carrier. In some aspects, oxytocin analogs can be formulated for intravenous administration. In some aspects, oxytocin analogs can be formulated for intramuscular administration. In some aspects, oxytocin analogs can be formulated for intranasal administration. In some aspects, oxytocin analogs can be formulated for sustained release administration. In some aspects, the sustained release formulation can be implantable. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes oxytocin dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

Also disclosed herein, are compositions, comprising oxytocin or analogs thereof and an opioid. In some aspects, the compositions can further include a pharmaceutical acceptable carrier. Compounding a formulation of oxytocin or oxytocin analogs with opioids can have numerous benefits. For example, the opioid dosage can be titrated to be lower if it is compounded with oxytocin or an oxytocin analog, given that oxytocin receptor activation has analgesic effects. Importantly, high doses of opioids may be administered to a subject safely, given that the data disclosed herein demonstrates that oxytocin can prevent and reverse respiratory depression. Furthermore, the data disclosed herein shows that opioids may be safer for subjects at a higher risk of opioid-induced respiratory depression, such as those with sleep apnea.

Also disclosed herein, are compositions, comprising oxytocin or analogs thereof and an opioid receptor antagonist. In some aspects, the compositions can further include a pharmaceutical acceptable carrier. Compounding a formulation of oxytocin with opioid receptor antagonists can also have numerous benefits. For example, opioid receptor antagonists used for life-saving reversal of opioid-induced respiratory depression can cause immediate opioid withdrawal, reversing the effects of, the exogenous opioids, but also the endogenous opioids the body produces that are involved in pain, mood, respiration, etc. Compounding oxytocin or oxytocin analogs with opioid receptor antagonists can be used for reversal of opioid-induced respiratory depression. The data disclosed herein demonstrates that the administration of oxytocin can reverse opioid-induced respiratory depression, as can the current gold standard, opioid receptor antagonists. Compounding the oxytocin or an oxytocin analog can be used to reduce withdrawal effects because the non-opioid analgesic (e.g., oxytocin) can effectively reverse opioid-induced respiratory depression caused by the opioid that is already present in the subject's system with the added benefit of opioid antagonism, making it possible to reduce the dosage of opioid receptor antagonist. Additionally, compounding oxytocin or oxytocin analogs with opioid receptor antagonists can provide a useful treatment option for managing opioid dependence and treating opioid abuse. One of the most widely used treatments (Suboxone®) is a formulation of a partial opioid agonist (e.g., buprenorphine) combined with an opioid receptor antagonist (e.g., naloxone). Buprenorphine has a ceiling effect for its effects on respiratory depression in most patients, but at-risk subjects with sleep apnea are still at risk for opioid-induced respiratory depression by opioids that are generally considered safer. There is also a stigma associated with suboxone treatment because its efficacy involves the taking of an opioid to treat opioid abuse and misuse. Buprenorphine binds with a higher affinity to opioid receptors than naloxone, and, as a partial agonist, produces less activity at receptors. Having a high affinity and lower efficacy, using an opioid to block the body's endogenous opioid system can produce imbalanced opioid signaling. However, oxytocin receptor activation, which is known to reduce opioid self-administration in rodents, could be formulated with opioid receptor antagonists for the treatment of opioid abuse and misuse or detoxification. Oxytocin or an oxytocin analog can act to reduce opioid seeking, and by combining with an opioid receptor antagonist at a lower dose can reduce the impairment of endogenous opioid actions while also acting as a safety net, together with oxytocin, to prevent opioid-induced respiratory depression.

Articles of Manufacture

The compositions described herein can be packaged in a suitable container labeled, for example, for use as a therapy to treat or prevent opioid toxicity or opioid overdose or opioid abuse or opioid-induced respiratory depression or opioid withdrawal symptoms or substance use disorder management or detoxification. Accordingly, packaged products (e.g., sterile containers containing the composition described herein and packaged for storage, shipment, or sale at concentrated or ready-to-use concentrations) and kits, including at least oxytocin or an oxytocin analog thereof as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1: Oxytocin Efficacy Against Opioid-Induced Respiratory Depression

The term opioids refers to substances that act on opioid receptors. Opioids can be compounds synthesized in the body, termed endogenous opioids, or analgesic drugs used under physician supervision such as fentanyl, oxycodone, hydrocodone, codeine, and morphine, or used illicitly including fentanyl and the illegal drug heroin. Opioid medications are the gold standard for acute clinical pain management. Despite their analgesic efficacy, opioids carry significant risk for lethal opioid-induced respiratory depression (OIRD), which largely accounts for the opioid overdose epidemic in the United States. Due to factors such as prior opioid use, drug-drug interactions, and comorbid conditions, OIRD sensitivity and severity is largely unpredictable. Over 130 people in the US die from opioid overdose daily with a large percentage of overdoses involving prescription opioids at a rate of 6.1 per 100,000 and 4.2 per 100,000 in men and women, respectively. Despite decreases from a peak prescription rate per 100 persons of 81.3 in 2012 to 58.5 in 2017, opioids are still one of the most widely prescribed medications in the U.S. according to CDC data. Given that prescription opioids will continue to play an important part of pain management for the foreseeable future, options that can reduce the risk of death are in high demand.

Current emergency intervention to treat OIRD involves the use of Narcan (naloxone). Naloxone competes with opioids for binding to opioid receptors with a high affinity, and once administered can lead to the reversal of OIRD in 2 minutes if given intravenously or 5 minutes if given intramuscularly. The issue with this rescue intervention is that opioid antagonism by naloxone does not discriminate between the opioid receptors that underlie OIRD and those responsible for analgesia, and consequently opioid-mediated analgesia is also reversed following naloxone rescue of OIRD. Therefore, there is a clear medical need for a way to combat the potentially lethal effects of OIRD, while maintaining the powerful analgesic effects of opioids.

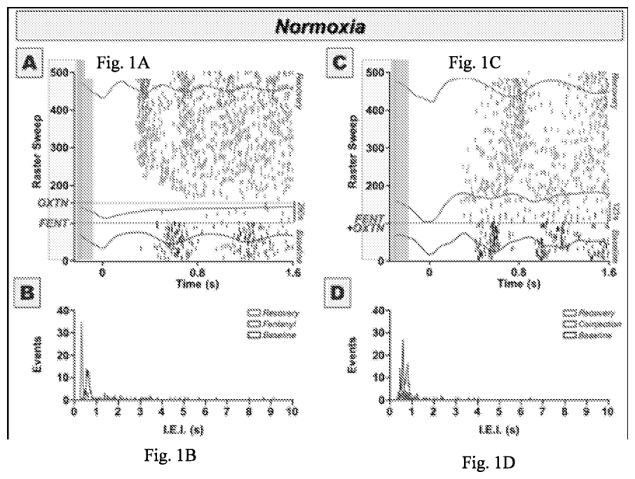

Upon systemic naloxone administration, neurons of the hypothalamic paraventricular nucleus (PVN) release the neuropeptide oxytocin into the blood stream via the posterior pituitary. Oxytocin release from the PVN has been demonstrated to have analgesic properties. This neuropeptide, which is FDA-approved for other indications, has been shown to participate in cardiorespiratory homeostasis in humans in conditions where oxytocin is believed to be depleted. Although oxytocin has never before been implicated in OIRD or rescue by naloxone, experiments were carried out to determine whether administration of the analgesic neuropeptide oxytocin may work similarly to naloxone. Indeed, systemic administration of oxytocin was found to rescue and attenuate OIRD by a lethal dose of the opioid fentanyl in conscious, unrestrained rats (FIG. 1).

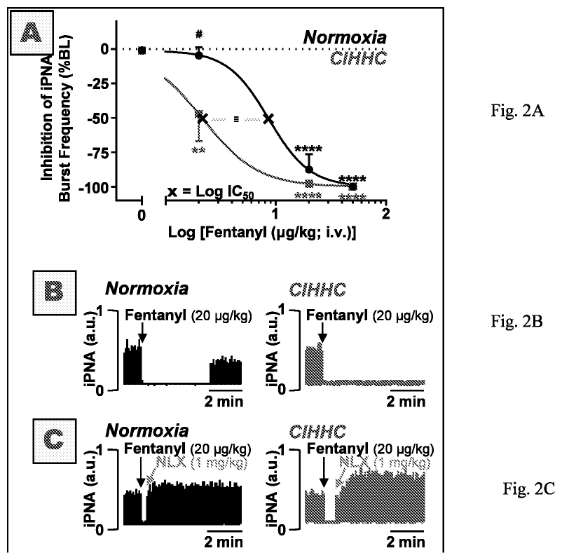

Next, experiments were carried out to determine whether oxytocin could also rescue and/or attenuate OIRD in a rat model of OIRD hypersensitivity. As mentioned herein, comorbid conditions make OIRD severity difficult to predict. One of the best predictors of OIRD is sleep apnea. Sleep apnea patients more frequently suffer fatal OIRD, and they more often require postoperative respiratory resuscitation with naloxone as well. Thus, a model for sleep apnea-related OIRD was established in which rats experienced chronic intermittent hypercapnic hypoxia (CIHHC). CIHHC exposure caused rats to become hypersensitive to fentanyl inhibition of inspiratory motor output. This is evident in FIG. 2A as a left-shift of the systemic fentanyl dose-phrenic nerve activity inhibition curve shown. Importantly, fentanyl OIRD was reversible by the opioid receptor antagonist naloxone (FIG. 2B-C).

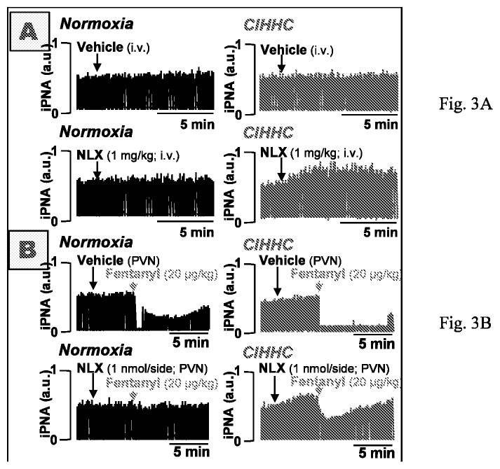

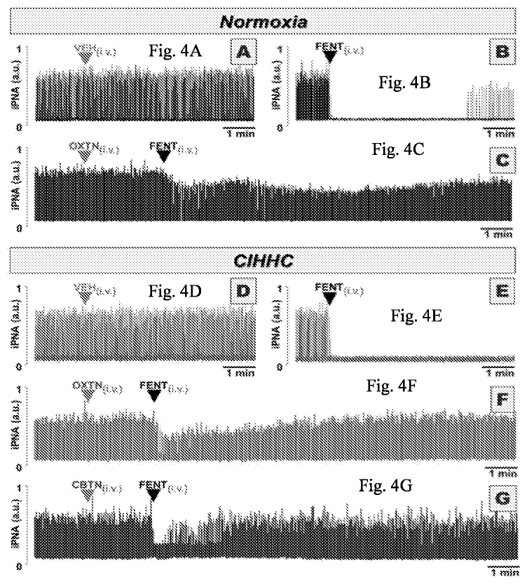

In rats with sleep apnea-related OIRD hypersensitivity, systemic injection of naloxone increases drive for inspiration (FIG. 3). Injection of naloxone directly into the hypothalamic PVN, where neurons synthesize oxytocin, can also prevent fentanyl OIRD. Like the conscious, unrestrained rat, systemic administration of oxytocin in the anesthetized rat also attenuated fentanyl OIRD, and this action occurs both in normoxic rats, as well as CIHHC rats that are more sensitive to OIRD by fentanyl (FIG. 4). Additionally, the oxytocin analog carbetocin, which is heat-stable—and has a longer half-life compared to oxytocin, —also attenuated fentanyl OIRD when administered systemically.

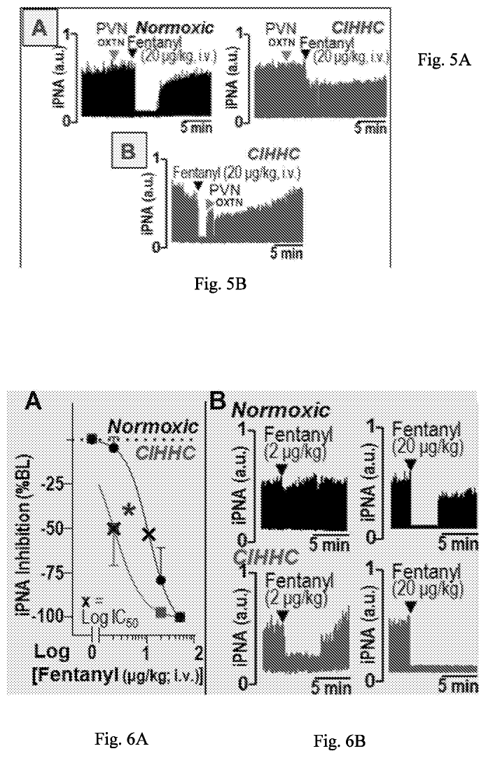

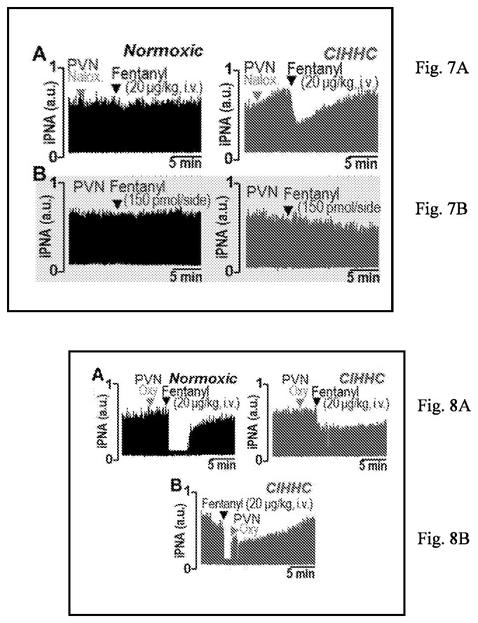

As mentioned herein, systemic naloxone administration induces the release of oxytocin from the PVN. Furthermore, systemic naloxone (FIG. 3) and oxytocin (FIG. 4) can attenuate fentanyl OIRD in CIHHC rats. Given that PVN antagonism of endogenous opioids attenuates OIRD, the ability of PVN oxytocin to mimic the effect of PVN naloxone was determined. Indeed, PVN administration of oxytocin attenuated and rescued OIRD by fentanyl in this model of OIRD hypersensitivity (FIG. 5). Given that a selective microinjection of oxytocin into the brain was capable of reversing OIRD, FIG. 6 shows a more translational approach with the systemically administered OXTN following fentanyl OIRD. Systemic OXTN effectively reversed fentanyl OIRD. The OXTN dose-response curve was bell-shaped, such that high doses of OXTN no longer reversed fentanyl OIRD. However, a vasopressin receptor antagonist facilitated OXTN reversal of fentanyl OIRD, showing that even the highest dose of OXTN tested was capable of full reversal of OIRD. Furthermore, OXTN receptor antagonism prevented OXTN reversal of OIRD. Finally, a selective OXTN agonist/vasopressin antagonist, WAY-267,474, was tested, which mimicked the results of OXTN in the presence of the vasopressin antagonist. These data show that systemic oxytocin in the absence of vasopressin receptor cross-activation reverses fentanyl OIRD in rats. Additional experiments demonstrated that a dose double the highest concentration used in rats, effectively reverses fentanyl OIRD in a second species, mice (FIG. 7).

These findings show using systemic oxytocin and its analogs, including non-peptidic orally active oxytocin receptor agonist compounds, as preventative treatments for OIRD as well as overdose resuscitation without compromising opioid-mediated analgesia, a role not currently fulfilled by naloxone. Using oxytocin and oxytocin analogs in combination with or compounded with opioids and opioid receptor antagonists would allow oxytocin receptor activation, which was recently demonstrated to be analgesic, and would be expected to improve current treatments for pain by, not only reducing the dosage of opioid analgesics, but most importantly by providing an improved safety profile to opioids. Furthermore, the oxytocin can reduce opioid self-administration preclinically and an oxytocin analgesic's ability to prevent and reverse opioid-induced respiratory depression could replace the opioid component in current opioid detoxification treatment (Suboxone®) if taken together or compounded with an opioid receptor antagonist. Additionally, oxytocin or oxytocin analogs used in combination or compounded with the opioid receptor antagonist that is clinically used can be titrated down to levels that would be less likely to produce withdrawal effects, while maintaining the life-saving respiratory depression reversal component.

The studies show a mechanism that involves the PVN, where oxytocin is synthesized.

The current standard of care for opioid overdose or OIRD is treatment with the opioid receptor antagonist naloxone, which competes for binding to opioid receptors. Once naloxone binds and displaces an opioid, it rapidly (2-5 min depending on administration method) leads to a reversal of both OIRD and analgesia. This is a problem in many medical settings, where the loss of analgesic effects results in an unacceptable level of pain and suffering to the patient.

Oxytocin is a neuro-peptide produced in the hypothalamus of the brain, and is implicated in a wide variety of social and non-social behaviors and activities that include “[ . . . ] social memory, attachment, sexual activity, maternal behavior, aggression, pair bonding, and trust. The nonsocial behaviors include brain development, learning and memory, feeding, respiration, cardiovascular activity, digestion, energy balance, thermoregulation, natriuresis, endocrine, immune regulation, pain perception, tolerance and dependence, autonomic outflow [ . . . ] lactation, and parturition.” Oxytocin has also been shown to participate in cardiorespiratory homeostasis in patients with sleep apnea. Although oxytocin is approved by the FDA and used medically during birth (parturition) to induce labor, speed labor, and stop bleeding after delivery, it is relatively cheap to produce. Oxytocin can have the structure represented by a formula:

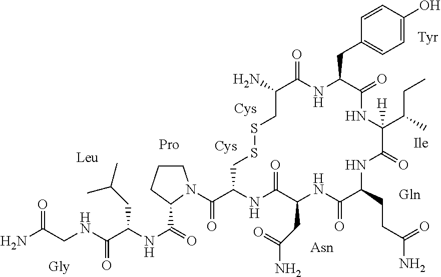 [SMILES: CC[C@H](C)[C@@H]1NC(=O)[C@H](Cc2ccc(O)cc2)NC(=O)[C@@H](N)CSSC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

Compared to the current standard of care for OIRD and opioid overdose, treatment with oxytocin has the following benefits but not limited to maintaining analgesia for a patient which current resuscitation drug naloxone cannot do, it is itself an analgesic which may allow for reduced amount of opioids to be used for the same amount of pain relief, may reduce opioid withdrawal symptoms, reduces opioid self-administration (Kovacs G L, et al. Life Sci. 1985 Jul. 8; 37(1):17-26), anti-depressant, FDA approved (for aiding in childbirth), and favorable side-effect profile.

Oxytocin has been shown to be effective both at preventing OIRD when given systemically before fentanyl, as well as reversing OIRD when oxytocin is given after fentanyl in both normoxic control rats and in rats modelling OIRD hypersensitivity. As described herein, the effectiveness of intra-nasal delivery of oxytocin was tested for its ability to prevent and reverse OIRD, as well as to investigate mechanisms that underlie the protective effect oxytocin has against OIRD.

Respiratory depression is indexed as reduced or absent bursting of phrenic nerve discharge. Delivery of the neuropeptide hormone oxytocin either into the systemic circulation or into a specific region of the brain, the paraventricular nucleus of the hypothalamus, can REDUCE or PREVENT (dose-dependent) respiratory depression caused by systemic administration of commonly prescribed opioid analgesics such as fentanyl and buprenorphine. Importantly, opioid-induced respiratory depression (OIRD) can also be REVERSED (breathing restored) after OIRD by subsequent administration of oxytocin into the systemic circulation, which is most effective in the presence of vasopressin receptor antagonism.

Use of opioids can result in lethal overdose caused by respiratory depression, but mechanisms of opioid induced respiratory depression (OIRD) remain to be elucidated. The currently available FDA approved treatment for OIRD is the opioid receptor antagonist naloxone (Narcan®). The foremost limitation of using naloxone to combat OIRD is that it effectively interrupts the pain killing (analgesic) action of opioids, including Fentanyl, buprenorphine and morphine. The compositions and methods disclosed herein can overcome these limitations by preventing and reversing OIRD while preserving the pain killing efficacy of commonly prescribed opioid pain killers. Studies revealed that the neuropeptide oxytocin (OXTN), which by itself reduces pain perception is effective both in reducing/preventing as well as reversing OIRD. One of the most vulnerable populations to suffer lethal OIRD are patients with sleep apnea (SA) (Ramachandran S K et al. 2011). While studying the underlying cause of increased OIRD sensitivity in SA led to the finding that OXTN acts as an effective treatment against OIRD. In regard to SA, it is noteworthy that patients with SA have been reported to have elevated cerebrospinal fluid (CSF) levels of endogenous opioids (Gislason T et al., 1989), which raised the possibility that combined actions of endogenous and exogenous opioids constitute a “double hit” that explains greater OIRD vulnerability in SA patients. This possibility is consistent with evidence that treatment with the opioid receptor antagonist naloxone improves symptoms of SA (Atkinson R L et al., 1985; Feber et al., 1988; Sonka K et al., 1989; Myer E C et al., 1990; Greenberg H E et al., 1991) as well as being effective against OIRD irrespective of whether patients do or do not suffer from SA.

In anesthetized rats, direct recordings of electrical nerve impulses from the phrenic nerve, i.e., phrenic nerve activity (PNA) was performed. The phrenic nerve is the primary nerve in mammalian species, including humans, that causes rhythmic contraction of the diaphragm muscle and hence inspiration/inhalation. Overall, data reveal that OXTN is effective against OIRD both when administered to normoxic control rats as well as rats exposed to chronic intermittent hypoxia with hypercapnia (CIHHC), which is an established animal model of human SA.

Evidence of OXTN efficacy against OIRD. FIG. 6 shows the phenomenon of OIRD and its enhancement by sleep apnea. The data illustrate graded OIRD induced by systemic administration of graded doses of the synthetic opioid Fentanyl. These data demonstrate that Fentanyl dose-dependently inhibits brain-driven neural inspiration, an effect exaggerated in a rat model of SA.

FIG. 7 shows that in a normoxic control rat (FIG. 7A, left) injection of the opioid receptor antagonist naloxone into the hypothalamic paraventricular nucleus (PVN), a brain region capable of stimulating respiration, has no effect. By contrast, the same dose of naloxone injected into the PVN of a CIHHC rat modeling SA (FIG. 7A, right) dramatically increases PNA (neutrally-driven inspiration). These findings indicate that CIHHC/SA increases endogenous opioid receptor inhibitory tone in the PVN. Failure of PVN naloxone to increase PNA burst amplitude in the normoxic control animal, indicates that PVN neurons are not normally inhibited by endogenous opioids but become inhibited upon exposure to CIHHC/SA. FIG. 7B (bottom) shows that a dose of PVN-injected Fentanyl that does not reduce PNA burst amplitude in a normoxic control animal (left), causes a small and gradual reduction of PNA bursting in a CIHHC exposed animal (right). This observation is consistent with clinical evidence from studies in SA patients and indicates that PVN neurons contribute to basal respiratory drive in this animal model of SA whereas these same neurons do not contribute to respiratory drive in normoxic controls. Collectively, these findings indicate that CIHHC increases endogenous opioid inhibition of PVN driven inspiration and makes animals more sensitive to opioid inhibition as well. Together, this “double-hit” may help explain increased vulnerability of SA patients to OIRD.

The results shown in FIG. 8A indicate that the neuropeptide OXT delivered directly into the PVN does not reduce OIRD in a normoxic control rat (left). By contrast, PVN OXY effectively blunts fentanyl-induced OIRD in a CIHHC-treated rat (right) (compared to FIG. 1A, right). Importantly, FIG. 8B shows in a CIHHC rat modeling SA that PVN OXT rapidly and completely reverses the arrest of breathing caused by systemic fentanyl.

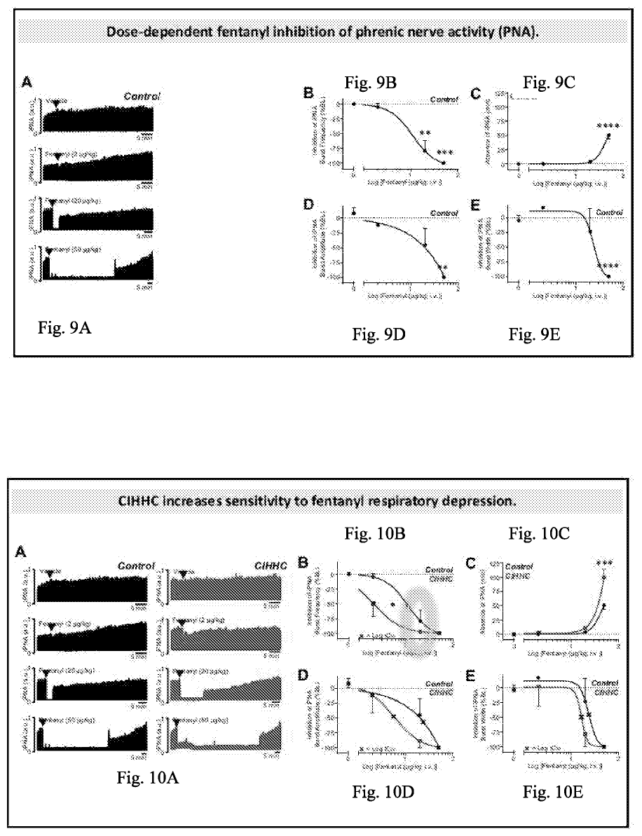

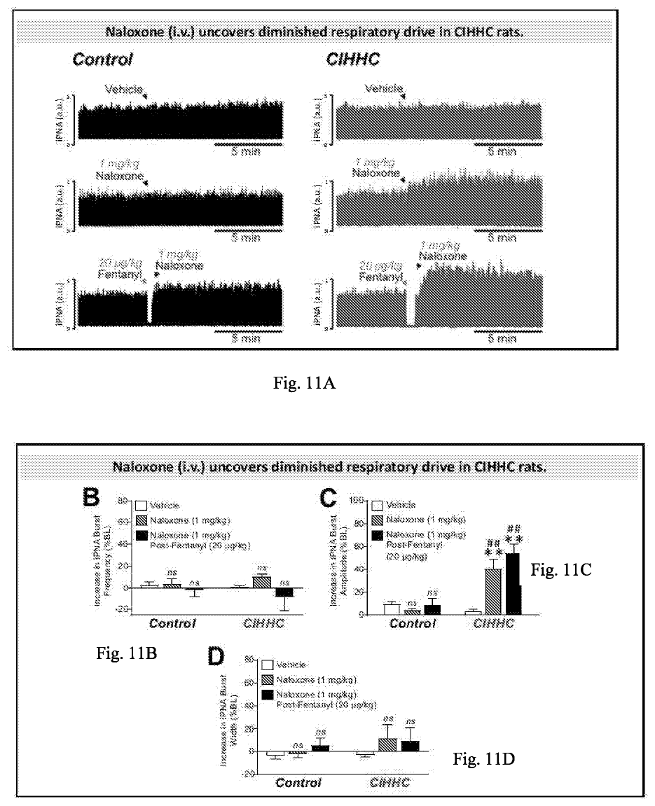

FIG. 9 shows that fentanyl dose-dependently inhibits phrenic nerve activity (PNA). FIG. 10 shows that CIHHC increases sensitivity to fentanyl respiratory depression. FIG. 11 shows that naloxone (i.v.) uncovers diminished respiratory drive in CIHHC rats.

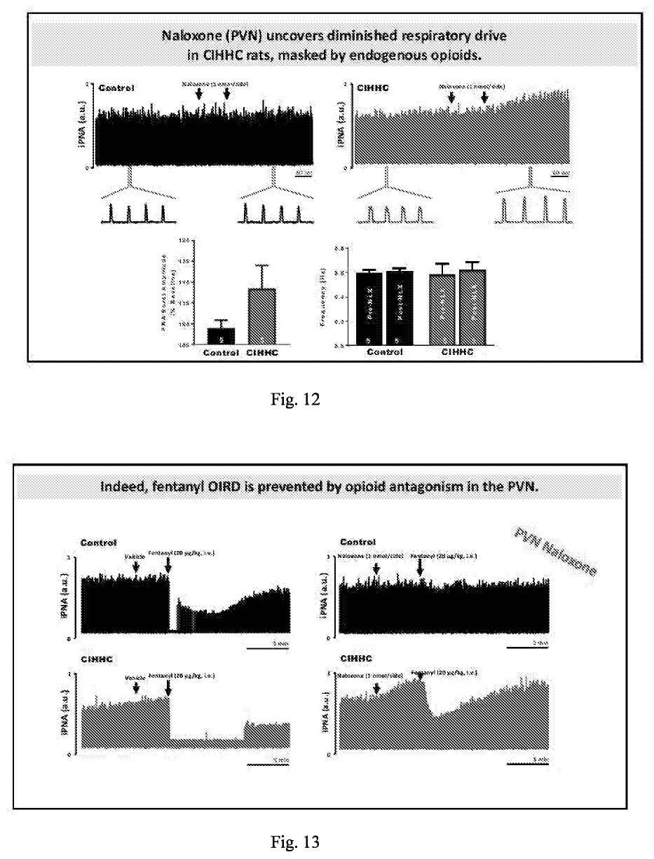

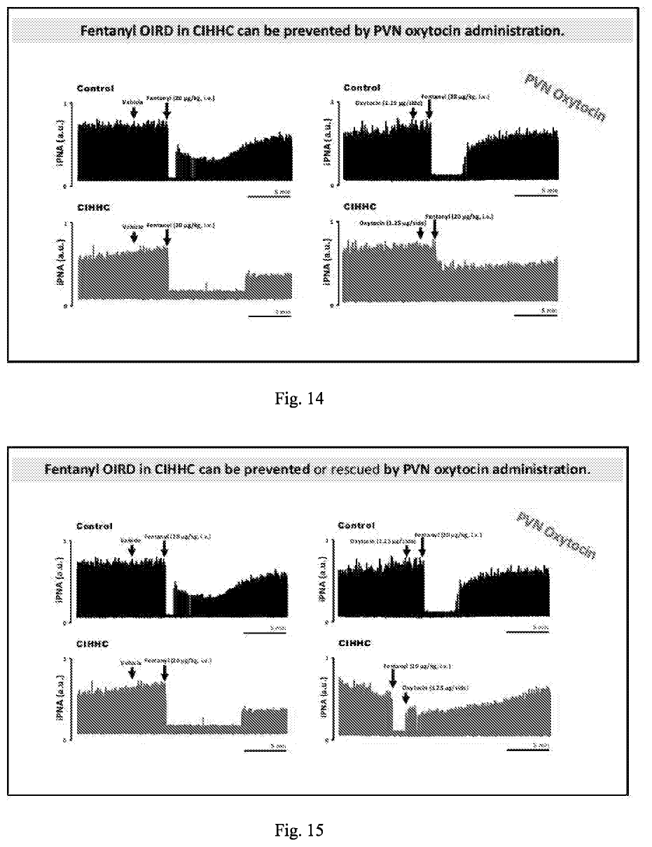

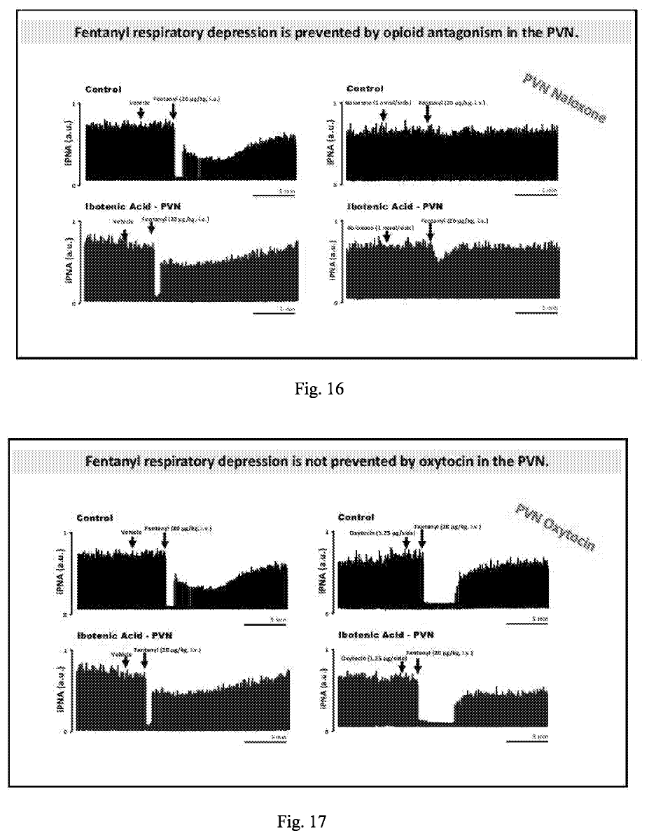

The next set of experiments tested whether increased endogenous opioids in the PVN inhibit oxytocin neurons and contribute to OIRD sensitivity in sleep apnea. FIG. 12 shows that naloxone into the PVN uncovers diminished respiratory drive in CIHHC rats, masked by endogenous opioids. FIG. 13 shows that fentanyl OIRD is prevented by opioid antagonism in the PVN. FIG. 14 and FIG. 15 show that fentanyl OIRD in CIHHC can be prevented and rescued by PVN oxytocin administration. FIG. 16 shows that fentanyl respiratory depression is prevented by opioid antagonism in the PVN. FIG. 17 shows that fentanyl respiratory depression is not prevented by oxytocin in the PVN.

These data show that similar to human sleep apnea patients, CIHHC rats may have increased CSF opioids as well as a differential sensitivity to clinically relevant opioids (e.g., fentanyl). In the rat model of sleep apnea, OIRD can be prevented by opioid antagonism in the PVN. Also, PVN oxytocin administration is protective against OIRD in CIHHC rats. Contrary to available literature evidence, which suggest that the PVN is a minimal driver of respirator output, the PVN appears to have a contribution to respiratory drive that is capable of conferring protection against systemic OIRD.

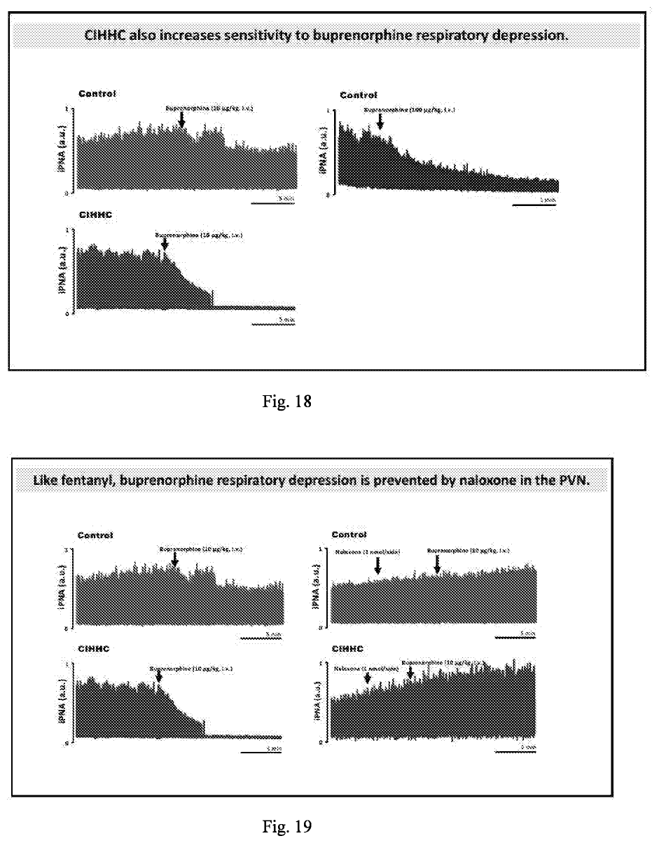

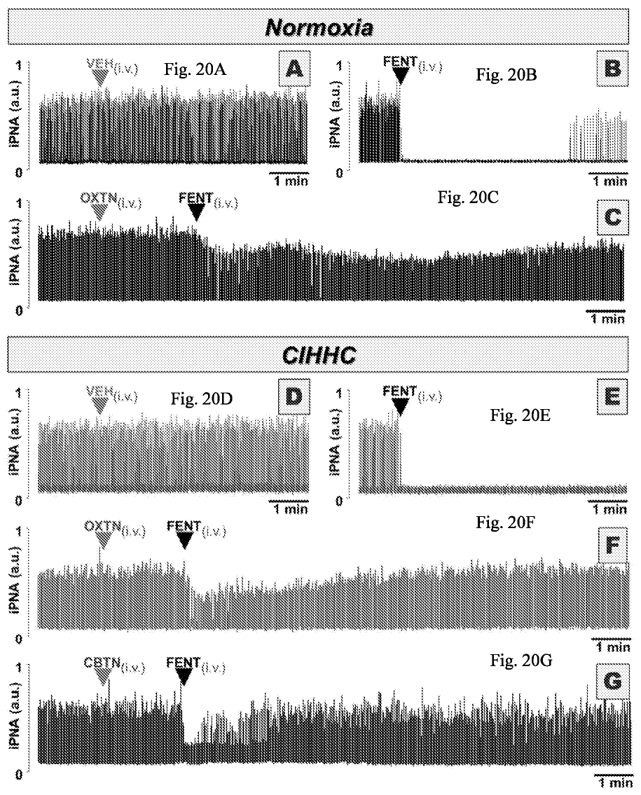

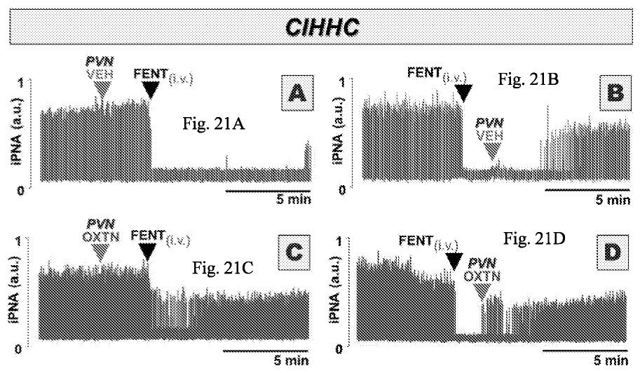

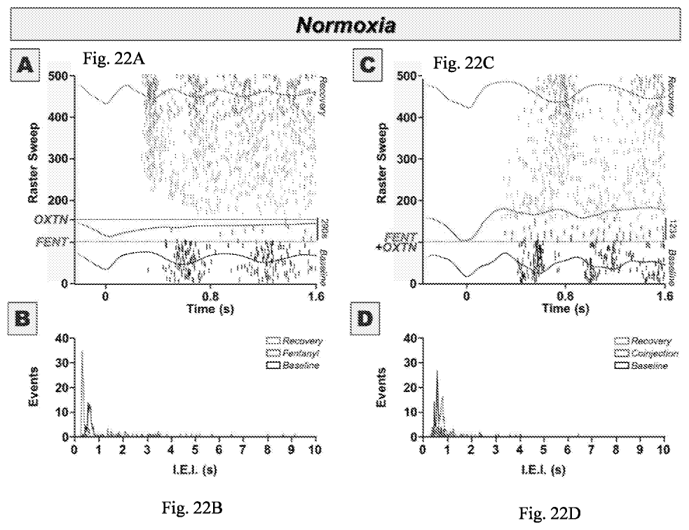

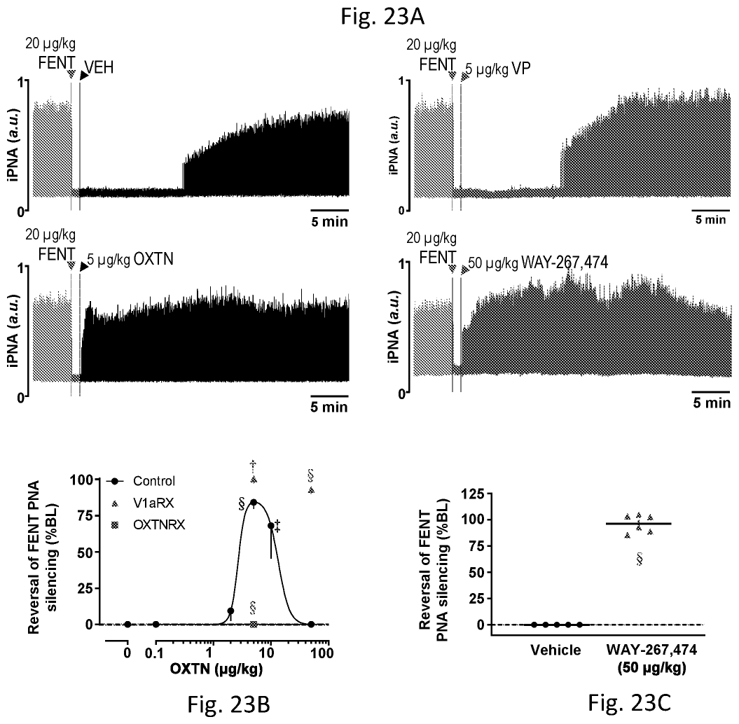

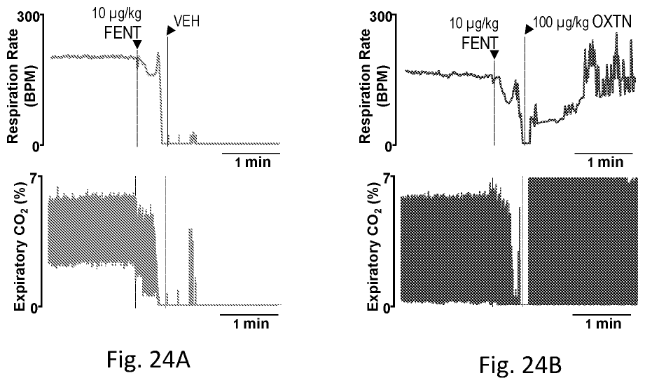

Additional experiments tested whether increased opioid onus in the PVN reduces dendritic release of oxytocin from magnocellular neurons. As a consequence, oxytocin parvocellular autonomic neurons that project to the respiratory network release less oxytocin making the respiratory network vulnerable to OIRD insult. FIG. 18 shows that CIHHC also increases sensitivity to buprenorphine respiratory depression. FIG. 19 shows that like fentanyl, buprenorphine respiratory depression is prevented by naloxone in the PVN. FIG. 20 shows that systemic oxytocin attenuates fentanyl OIRD in normoxic rats and CIHHC exposed rats that serve as a sleep apnea model of enhanced OIRD sensitivity. FIG. 21 shows that oxytocin can act in the hypothalamic paraventricular nucleus (PVN) to attenuate and rescue fentanyl-induced OIRD. FIG. 22 shows that systemic oxytocin can rescue and attenuate fentanyl OIRD in awake freely moving rats. For these experiments, conscious, unrestrained rats that were surgically instrumented with wireless pressure transducer transmitters to monitor subpleural thoracic pressure fluctuations caused by rhythmic breathing. Inspiratory phases of breathing were monitored before and after systemic (intravenous, i.v.) administration of a lethal dose of fentanyl (FENT; 100 μg/kg, i.v.). Fentanyl at an (100 μg/kg, i.v.) increased the inspiratory interval and disrupted it rhythmicity. This gradually worsened until oxytocin (150 μg/kg, i.v.) was administered at 290 s post-fentanyl to avoid lethality. Importantly, systemic oxytocin rescued lethal fentanyl OIRD in the freely moving rat and breathing recovered to overshoot baseline frequency. Co-administration of the same lethal dose of fentanyl (100 μg/kg, i.v.) in combination with oxytocin (150 μg/kg, i.v.) largely attenuated OIRD, with the same number of breaths that spanned 290 s post-fentanyl in panel A occurring in just 123 s. Systemic oxytocin was given at the same time as lethal fentanyl attenuated OIRD in the freely moving rat and breathing recovered nearly to baseline cadence.

What is claimed is:

1. A method of preventing, reversing or reducing opioid-induced respiratory depression in a subject, the method comprising: administering to the subject a therapeutically effective amount of oxytocin or an analog thereof and a pharmaceutically acceptable salt.

2. (canceled)

3. (canceled)

4. A method of preventing or reducing one or more symptoms of opioid withdrawal in a subject, the method comprising: administering to the subject a therapeutically effective amount of oxytocin or an analog thereof, an opioid receptor antagonist and a pharmaceutically acceptable salt.

5. The method of claim 1, wherein the subject has been diagnosed with or has sleep apnea or obstructive sleep apnea.

6. The method of claim 1, wherein the subject is undergoing opioid treatment for pain.

7. The method of claim 1, wherein the subject is identified in need of treatment before the administration step.

8. The method of claim 1, wherein the oxytocin or analog thereof is administered orally, intravenously, intramuscularly or intranasally.

9. The method of claim 1, wherein the oxytocin or analog thereof is administered on an as-needed basis.

10. The method of claim 1, wherein the therapeutically effective amount of oxytocin or the analog thereof is administered before, during or after administration of an opioid.

11. The method of claim 1, wherein the oxytocin analog is carbetocin or WAY-267,474.

12. A pharmaceutical composition comprising oxytocin or an analog thereof and an opioid.

13. The pharmaceutical composition of claim 12, wherein the oxytocin analog is carbetocin or WAY-267,474.

14. The pharmaceutical composition of claim 12, wherein the composition is formulated for oral, intravenous, intramuscular or intranasal administration.

15. The method of claim 1, further comprising the administration of an opioid receptor antagonist.

16. The method of claim 1, further comprising the administration of an opioid receptor antagonist.
